(19) United States
(12) Patent Application Publication
(10) Pub. No.: US 2008/0095842 A1
(43) Pub. Date: Apr. 24, 2008

(54) Rapidly Disintegrating Taste Masked Compositions and a Process for Its Preparations

(76) Inventors: Amit Krishna Antarkar, Maharashtra (IN); Hemant Manilal Mamania, Maharashtra (IN)

Correspondence Address: HOLLAND & BONZAGNI, P.C., 171 DWIGHT ROAD, SUITE 302, LONGMEADOW, MA, 01106-1700, US

(21) Appl. No.: 11/792,351

(22) PCT Filed: Nov. 29, 2005
(86) PCT No.: PCT/IB05/03707

§ 371 Date: Jun. 5, 2007

(30) Foreign Application Priority Data
Dec. 6, 2004 (IN) 1296/MUM/2004

(51) Int. Cl. A61K 31/495; A61K 9/20; A61P 43/00; C07D 241/04

(52) U.S. Cl. 424/465; 514/255.04; 544/396

(57) ABSTRACT

A resinate of Cetirizine or its pharmaceutically acceptable salts or its enantiomers or their salts such as Levocetirizine Dihydrochloride, fast disintegrating and or quick release pharmaceutical compositions containing the resinate and the process for the preparation of the said resinate and composition is disclosed. Preparation of resinate and composition comprising resinate is carried out preferably in aqueous solvents.

### FIELD OF INVENTION

The present invention relates to resinates of Cetirizine or its pharmaceutically acceptable salts or its enantiomers or pharmaceutically acceptable salts of its enantiomers such as Levocetirizine Dihydrochloride, fast disintegrating and or quick release pharmaceutical compositions comprising the said resinates and process for the preparation of resinates and compositions.

### BACKGROUND OF THE INVENTION

Oral dosage forms containing Cetirizine or its pharmaceutically acceptable salts or its enantiomers or pharmaceutically acceptable salts of its enantiomers such as Levocetirizine Dihydrochloride have bitter taste and therefore need to be taste masked. Taste masking is generally done by various means such as formation of new salt, coating, inclusion complexes, resinates, etc to enable oral dispersion of compositions. that are “fast melt” or “rapid melt” or “quick disintegrating”, which are prepared using technologies such as Zydis, Orasolv, Flashdose.

Issues related to resinate formation relate to choosing the appropriate resin as it is drug specific which in turn strongly influence the drug release profile, drug stability in the resinate and resinate dispersibility.

U.S. Pat. No. 2,990,332 discloses a method for loading active substances onto an ion exchange resin, loading being dependant on the rate of diffusion, the equilibrium constant, temperature, and the presence of other ions.

U.S. Pat. No. 6,565,877 teach a taste masked composition which comprises a bitter tasting drug, a combination of two .enteric polymers comprising, a methacrylic acid copolymer and a phthalate polymer. The patent cautions on the use of cation—exchange resins such as polysulfonic acid and polycarboxylic acid polymers to adsorb amine drugs for taste masking as the drug release may be compromised.

U.S. Pat. No. 5,071,646 entitled “Pharmaceutical Ion-exchange Resin Composition”, discloses resin compositions comprising a granulated ion-exchange resin, a pharmacologically active ingredient bound thereto with a sugar or sugar alcohol, and a sufficient amount of water, alcohol or aqueous alcohol to facilitate granulation and the ratio of active agent to ion-exchange resin may vary between about 1:3 to 2:1 such that the compositions are dispersible in large quantity of water, twenty times the weight of composition when stirred.

U.S. Pat. No. 5,188,825 discloses freeze-dried dosage forms prepared by bonding or complexing a water-soluble active agent to or with an ion exchange resin to form a substantially water insoluble complex.

U.S. Pat. No. 5,219,563 teach use of synthetic cation exchange resins such as copolymers of styrene and divinylbenzene which are sulphonated and copolymers of methacrylic acid and divinylbenzene for masking the bitter taste of ranitidine.

Sriwongjanya M, Bodmeier R. Eur J Pharm Biopharm. 1998 November; 46(3):321-7 teaches that the drug release from HPMC matrix based tablets containing drug-resin complexes is significantly slower than from HPMC matrix based tablets containing drug without resin.

The Publications “Fast-Dissolving Tablets” in US Pharmacist March 2002 and “Design of Fast Dissolving Tablets “IJPE—Indian Journal of Pharmaceutical Education, October-December, 2001” indicate following drawbacks of technologies related to fast dissolving tablets using non-aqueous solvents:

1) Tablets produced by Zydis technology are soft, fragile and therefore not suitable for conventional blister packing. These are hygroscopic and exhibit poor stability during test conditions.

2) Tablets produced by orasolv technology are soft and friable and hence packaged using an integrated packaging line that uses a specially designed robotic pick and pack system.

3) A specialized packaging is required to protect highly friable, soft and moisture sensitive tablets of Flashdose technology.

It is a longstanding need of the industry to provide resinates of Cetirizine or its pharmaceutically acceptable salts or its enantiomers or pharmaceutically acceptable salts of its enantiomers such as Levocetirizine Dihydrochloride having taste masking properties and their compositions that are rapid disintegrating and or quick release, optionally containing other actives. Further it is desirable to prepare such compositions without the use of nonaqueous solvents thereby obviating the use of sophisticated machinery as is done in prior art.

### OBJECTS OF THE INVENTION

The main object of the invention is to provide resinates of Cetirizine or its pharmaceutically acceptable salts or its enantiomers or pharmaceutically acceptable salts of its enantiomers such as Levocetirizine Dihydrochloride, rapid disintegrating and or quick release taste masked pharmaceutical compositions comprising the said resinates and process for the preparation of resinates and compositions.

Another object of the invention is to prepare stable resinates of Cetirizine or its pharmaceutically acceptable salts or its enantiomers or pharmaceutically acceptable salts of its enantiomers such as Levocetirizine Dihydrochloride, rapid disintegrating and or quick release taste masked pharmaceutical compositions comprising the said resinates and process for the preparation of resinates and compositions.

Yet another object of the invention is to provide easily dispersible resinates of Cetirizine or its pharmaceutically acceptable salts or its enantiomers or pharmaceutically acceptable salts of its enantiomers such as Levocetirizine Dihydrochloride, rapid disintegrating and or quick release taste masked pharmaceutical compositions comprising the said resinates and process for the preparation of resinates and compositions.

Yet another aspect of the invention is to provide a rapidly disintegrating taste masked composition of Cetirizine or its pharmaceutically acceptable salts or its enantiomers or pharmaceutically acceptable salts of its enantiomers such as Levocetirizine Dihydrochloride without the use of non-aqueous solvents thereby obviating the use of sophisticated machinery.

Yet another object of the invention to provide taste masked rapidly disintegrating and or quick release composition of Cetirizine. or its physiologically acceptable salt or its enantiomers or salts thereof or Levocetrizine Dihydrochloride with at least one more active ingredient.

### SUMMARY OF INVENTION

The present invention relates to stable and readily dispersible resinates of Cetirizine or its pharmaceutically acceptable salts or its enantiomers or pharmaceutically acceptable salts of its enantiomers such as Levocetirizine Dihydrochloride, rapid disintegrating and or quick release taste masked pharmaceutical compositions comprising the said resinates and process for the preparation of resinates and compositions. Further the invention relates to the preparation of resinates and the compositions comprising the resinates without the use of non-aqueous solvents thereby obviating the use of sophisticated packaging machinery.

The process for preparation of resinates and compositions with resinate comprises steps:

1) Contacting solution of Cetirizine or its pharmaceutically acceptable salts or its enantiomers or pharmaceutically acceptable salts of its enantiomers such as Levocetirizine Dihydrochloride with ion exchange resin at pH 0.5-8,

2) Separating the resinate and drying it at temperatures below 115° C.,

3) Optionally processing the resinate to obtain fast disintegrating and or quick release compositions with or without other actives.

### DESCRIPTION OF THE INVENTION

The process of preparation of resinate and composition comprises steps of:
a) Dissolving Cetirizine or its pharmaceutically acceptable salts or its enantiomers or pharmaceutically acceptable salts of its enantiomers such as Levocetirizine Dihydrochloride in an appropriate solvent;
b) Adjusting the pH to 0.5-8;
c) Contacting ion exchange resin with the solution obtained in step b;
d) Washing the resinate with solvent followed by drying the resinate;
e) The dried resinate is optionally processed to obtain fast disintegrating and or quick release compositions.

The resinate is processed to obtain oral dosage compositions such as quick dispersing tablets, granules for dispersion either in sachet pack or as a dry syrup, dispersed into flavored syrupy base for liquid suspension, mixed with chewing gum base for chewing gum composition, dispersed into lozenge base to have a lozenge, with cooked glucose or similar base for having a lollypop, into a wafer base or a soluble film base to have medicated soluble film or wafer.

The efficiency of drug loading achieved in preparation of resinate is not less than 95%.

The cation exchange resin used for resinate formation is selected from sulphonated copolymers of styrene and divinylbenzene, or copolymers of methacrylic acid and divinylbenzene, cross linked polymer of methacrylic acid and divinylbenzene including those available commercially as Dowex resins, Amberlite IRP resins, Tulsion resins Indion resins and their equivalents in acid form or in the form of salt with alkali metals. The ratio of resin-to drug is in the range of 5:1 to 0.1:1, preferable being 3:1 to 0.5:1, more preferable being 2:1 to 1:1.

Solvent and or co-solvent is selected from water, ethanol, organic polar and non-polar solvents, glycerin, propylene glycol, polyethylene glycol and their suitable mixture. Preferred solvent is one in which the active ingredient has substantial solubility.

Process for drying is selected from evaporation, vacuum evaporation, tray drying, oven drying, air drying at room or elevated temperatures, microwave drying, spray drying, drum and belt film drying; or by centrifuging and optionally followed by drying or by other suitable method. Drying of resinate is carried out at a temperature less than 115° C. It is advisable to carry out drying at temperature range of about 20° C. to about 114° C., preferable range being 30° C. to 90° C., more preferable range being 40° C. to 80° C., most preferable range being 50° C. to 70° C.

Active ingredient is Cetirizine or its pharmaceutically acceptable salts or its enantiomers or pharmaceutically acceptable salts of its enantiomers such as Levocetirizine Dihydrochloride.

The content of active ingredient in the resinate is upto 50% w/w, preferably from 10% to 45% w/w and more preferably form 20% to 40% w/w and most preferably from 30% to 35% w/w of that of resinate.

The resinate is optionally processed with pharmaceutically acceptable excipients to obtain fast disintegrating and or quick release compositions such as quick dispersing tablets, sachet pack of granules for dispersion or as a dry syrup, dispersed into flavored syrupy base for liquid suspension, mixed with chewing gum base for chewing gum composition, dispersed into lozenge base to have a lozenge, with cooked glucose or similar base for having a lollypop, into a wafer base or a soluble film base to have medicated soluble film or wafer by the processes known to the persons skilled in the art. Further the compositions containing resinates may optionally contain one or more additional actives.

The resinate was given to 6 volunteers for testing palatability. The resinate was judged to be substantially free from bitter taste otherwise associated with Cetirizine or its pharmaceutically acceptable salts or its enantiomers or pharmaceutically acceptable salts of its enantiomers such as Levocetirizine Dihydrochloride. The compositions comprising resinate were given to 6 volunteers for testing palatability, mouth-feel, and taste. It was also reported that the compositions were substantially free from the bitter taste, otherwise associated with Cetirizine or its pharmaceutically acceptable salts or its enantiomers or pharmaceutically acceptable salts of its enantiomers such as Levocetirizine Dihydrochloride. The disintegration time of the tablet in the oral cavity was about 30-45 seconds.

The invention is illustrated with non-limiting examples.

### EXAMPLE 1

### Taste Masked Levocetirizine Resinate

50 gms of Levocetirizine Dihydrochloride was dissolved in 900 ml water. The pH of this solution was adjusted to about 6 using 2N sodium hydroxide solution in water. 100 gm of Tulsion 335 was slurred and stirred for 4 hours. The suspension was filtered through an appropriate filter and the solid was re-suspended in about 1000 ml water. Suspension obtained was stirred for about 12 hours. Stirred suspension was filtered using appropriate filters. This process of filtration and resuspension was repeated for 3 times. Finally the suspension was filtered through an appropriate filter and resinate was dried at 60° C. in tray dryer. The dried resinate contained about 32% w/w of Levocetirizine compared to theoretical 33.33% w/w of Levocetrizine at the proportion taken in this experiment. Yield as high as 98% w/w was achieved. As per the evaluation by volunteers said resinate was substantially free from bitter taste associated with drug.

### EXAMPLE 2

### Rapid Disintegrating Taste Masked Tablet Composition of Resinate of Levocetirizine.

| Ingredient | Qty/Batch (gm) | Mg/Unit |
|---|---|---|
| Levocetrizine resinate | 22.72 | 15.03 |
| Pearlitol SD200 | 146.06 | 98.59 |
| Crospovidone | 10.00 | 6.75 |
| Mg Stearate | 6.11 | 4.12 |
| Aerosil | 3.33 | 2.25 |
| Aspartame | 6.67 | 4.50 |
| Strawberry Flavor | 5.56 | 3.75 |

Resinate was mixed with diluent (Pearlitol SD200), disintegrants (crospovidone) lubricants (colloidal silicon dioxide and magnesium stearate), sweetener and flavor, after passing through suitable sieve. The resulting mixture was compressed into tablets so as to contain 5 mg of Levocetirizine base. Tablets had hardness of about 3 kps/cm², friability of 0.29% for 100 revolutions and were packed in Alu/Alu, Alu/PVC packing foils using conventional machines. The tablets rapidly disintegrate in mouth in not more than 60 seconds when tested in 10 volunteers and in USP disintegration test apparatus.

### EXAMPLE 3

### Taste Masked Resinate of Cetirizine Dihydrochloride

50 gm of Cetirizine dihydrochloride was dissolved in 900 ml water. The pH of this solution was adjusted to about 5.98 using 2N sodium hydroxide solution in water. 100 gm of Tulsion 335 was slurred and stirred for about 4 hours. The suspension was filtered through an appropriate filter and the solid was re-suspended in about 1000 ml water. Suspension obtained was stirred for about 12 hours. Stirred suspension was filtered using appropriate filters. This process of filtration and resuspension was repeated 3 times. Finally the suspension was filtered through an appropriate filter and resinate was dried at 60° C. Yield was about 98%. As per the evaluation by volunteers said resinate was substantially free from the bitter taste associated with drug.

### EXAMPLE 4

### Rapid Disintegrating Taste Masked Tablet Composition of Resinate of Cetirizine

| Ingredient | Qty/Batch (gm) | Mg/Unit |
|---|---|---|
| Cetirizine resinate | 30.45 | 30.45 |
| Pearlitol SD200 | 100.43 | 100.43 |
| Crospovidone | 4.50 | 4.50 |
| Mg Stearate | 4.13 | 4.13 |
| Aerosil | 2.25 | 2.25 |
| Aspartame | 4.50 | 4.50 |
| Pineapple Flavor | 3.75 | 3.75 |

Resinate was mixed with diluent (Pearlitol SD200), disintegrants (crospovidone) lubricants (colloidal silicon dioxide and magnesium stearate), sweetener and flavor, after passing through suitable sieve. The resulting mixture was compressed into tablets so as to contain 10 mg of Cetirizine base per tablet. By reducing the quantity of resinate proportionally we get tablets containing 5 mg of Cetirizine base per tablet. These tablets have hardness of about 3 kps/cm², friability of 0.37% for 100 revolutions and are packed in Alu/Alu Alu/PVC packing foils using conventional machines. The tablets rapidly disintegrate in mouth in not more than 60 seconds when tested in 10 volunteers and in USP disintegration test apparatus.

### EXAMPLE 5

### Composition Comprising Resinate of Levocetirizine with Sustained Release Dextromethorphan

Resinate of Levocetirizine as per example 1 was processed with the Dextromethorphan resinate equivalent to 10 mg of Dextromethorphan HBr per dose along with pharmaceutically acceptable ingredients as in example 2 to have a tablet composition.

### EXAMPLE 6

### Composition Comprising Resinate of Levocetirizine with Pseudoephedrine Sulphate

Resinate of Levocetirizine as per example 1 was processed with resinate of Pseudoephedrine Sulphate equivalent to 30 mg Pseudoephedrine Sulphate per unit dose along with pharmaceutically acceptable ingredients as in example 2 to have a tablet composition. Pseudoephedrine resinate wherein Pseudoephedrine is in extended release is also processed similarly. Pseudoephedrine resinate containing Pseudoephedrine 60 mg/120 mg/240 mg per unit dosage is also processed similarly.

### EXAMPLE 7

### Composition Comprising Resinate of Levocetirizine with Salbutamol Sulphate

Resinate of Levocetirizine as per example 1 is processed with controlled release micro-particles of Salbutamol Sulphate along with pharmaceutically acceptable ingredients as in example 2 to have a fast disintegrating tablets containing 2 mg of Salbutamol Sulphate per tablet as additional active ingredient.

### EXAMPLE 8

### Composition Comprising Resinate of Levocetirizine with Vitamin B₁₂

Resinate of Levocetirizine as per example 1 is processed with resinate of Vitamin B₁₂ equivalent to 100 microgram to 3000 microgram along with pharmaceutically acceptable ingredients as in example 2 and compressed to have a fast disintegrating tablet comprising Vitamin B₁₂ as additional active ingredient.

### EXAMPLE 9

### Dry Syrup Composition Comprising Resinate of Levocetirizine

| Ingredient | Qty/Batch (gm) | Mg/5 ml |
|---|---|---|
| Levocetrizine resinate | 1.90 | 15.83 |
| Sodium CMC | 1.00 | 8.33 |
| Sucrose | 0.95 | 7.91 |
| Propyl Paraben | 0.10 | 0.83 |
| Water to | 600 ml | |

Levocetrizine resinate 1.9 gms in 600 ml is equal to 5 mg of Levocetrizine per 5 ml. Levocetrizine resinate, Sodium CMC, Sucrose and Propyl Paraben were mixed in mixer. Water was added to make volume to 600 ml. Similarly in another experiment 600 ml were prepared without sugar. The compositions were studied for 7 days. Subjective evaluation testing was done during and after 7 days. Not only the composition containing sugar was substantially free from bitter taste, but also the composition without sugar was substantially free from bitter taste even after 7 days. The suspensions remain easily re-dispersible at the end of 7 days and were palatable.

### EXAMPLE 10

### Stability of Levocetirizine Resinate at 80° C. with Respect to Taste

Resinate when kept at 80° C. in hot air oven for 30 minutes, did not show any change with respect to its taste masking ability. It was substantially free from bitter taste associated with drug.

In the same manner one can have a composition comprising a resinate of Cetirizine or its pharmaceutically acceptable salts or its enantiomers or pharmaceutically acceptable salts of its enantiomers such as Levocetirizine Dihydrochloride and ion exchange resin with two or more active pharmaceutically acceptable ingredients selected from Pseudoephedrine, Paracetamol, Phenylpropanolamine, Caffeine, Ambroxol, Salbutamol, Phenylephrine, Vitamins like Vitamin B₁₂ or their pharmaceutically acceptable salts or their enantiomers or salts thereof.

1) A resinate of Cetirizine or its pharmaceutically acceptable salts or its enantiomers or pharmaceutically acceptable salts of its enantiomers such as Levocetirizine Dihydrochloride.

2) The resinate as claimed in claim 1, wherein the resin is selected from the group of copolymers of methacrylic acid and divinylbenzene, cross linked polymer of methacrylic acid and divinylbenzene, and sulphonated copolymers of styrene and divinylbenzene.

3) The resinate as claimed in claim 1, wherein the resin is used in its free acid form or in the form of alkali metal salt.

4) The resinate as claimed in claim 1, wherein Cetirizine or its pharmaceutically acceptable salts or its enantiomers or pharmaceutically acceptable salts of its enantiomers such as Levocetirizine Dihydrochloride is present in an amount up to 50% w/w, expressed as the weight of free base of Cetirizine or its enantiomers such as Levocetirizine, to the weight of resinate.

5) The resinate as claimed in claim 1, wherein the ratio of resin to Cetirizine or its pharmaceutically acceptable salts or its enantiomers or pharmaceutically acceptable salts of its enantiomers such as Levocetirizine Dihydrochloride is in the range of 5:1 to 0.1:1.

6) A process of preparation of resinate comprising the steps of:
(a) dissolving Cetirizine or its pharmaceutically acceptable salts or its enantiomers or pharmaceutically acceptable salts of its enantiomers such as Levocetirizine Dihydrochloride in an appropriate solvent to form a solution;
(b) adjusting the pH of the solution to 0.5-8;
(c) contacting ion exchange resin with the solution obtained in step b to obtain resinate;
(d) optionally washing the resinate with solvent followed by drying the resinate;
(e) optionally processing the dried resinate to obtain fast disintegrating and/or quick release compositions.

7) The process as claimed in claim 6, wherein the resin is used in its free acid form or in the form of alkali metal salt.

8) The process as claimed in claim 6, wherein the resin is selected from the group of copolymers of methacrylic acid and divinylbenzene, cross linked polymer of methacrylic acid and divinylbenzene, and sulphonated copolymers of styrene and divinylbenzene.

9) The process as claimed in claim 6, wherein Cetirizine or its pharmaceutically acceptable salts or its enantiomers or pharmaceutically acceptable salts of its enantiomers such as Levocetirizine Dihydrochloride is present in an amount up to 50% w/w, expressed as the weight of free base of Cetirizine or its enantiomers such as Levocetirizine, to the weight of resinate.

10) The process as claimed in claim 6, wherein solvent used is selected from the group of water, alcohol, pharmaceutically acceptable organic solvent, inorganic solvent and mixtures thereof.

11) The process as claimed in claim 6, wherein the ratio of resin to Cetirizine or its pharmaceutically acceptable salts or its enantiomers or pharmaceutically acceptable salts of its enantiomers such as Levocetirizine Dihydrochloride is in the range of 5:1 to 0.1:1.

12) The process as claimed in claim 6, wherein drying is carried out at a temperature ranging from about 20° C. to about 114° C.

13) A composition comprising a resinate as claimed in claim 1, wherein the resinate content is from 0.01% w/w to 100% w/w of the weight of composition.

14) A fast disintegrating tablet comprising a resinate of Cetirizine or its pharmaceutically acceptable salts or its enantiomers or pharmaceutically acceptable salts of its enantiomers such as Levocetirizine Dihydrochloride, wherein the resinate content is 0.01% w/w to 100% w/w of the weight of composition.

15) The composition as claimed in claim 13, further comprising pharmaceutically acceptable excipients optionally with one or more additional active ingredients.

16) The composition as claimed in claim 15, wherein the additional active ingredient present is selected from the group of Pseudoephedrine, Paracetamol, Phenylpropanolamine, Caffeine, Ambroxol, Salbutamol, Phenylephrine, Vitamins, their pharmaceutically acceptable salts, their enantiomers, the salts of their enantiomers, and mixtures thereof.

17) The resinate as claimed in claim 5, wherein the ratio of resin to Cetirizine or its pharmaceutically acceptable salts or its enantiomers or pharmaceutically acceptable salts of its enantiomers such as Levocetirizine Dihydrochloride is in the range of 2:1 to 1:1.

18) The process as claimed in claim 11, wherein the ratio of resin to Cetirizine or its pharmaceutically acceptable salts or its enantiomers or pharmaceutically acceptable salts of its enantiomers such as Levocetirizine Dihydrochloride is in the range of 2:1 to 1:1.

19) The fast disintegrating tablet as claimed in claim 14, further comprising pharmaceutically acceptable excipients optionally with one or more additional active ingredients.

20) The fast disintegrating tablet as claimed in claim 19, wherein the additional active ingredient present is selected from the group of Pseudoephedrine, Paracetamol, Phenylpropanolamine, Caffeine, Ambroxol, Salbutamol, Phenylephrine, Vitamins, their pharmaceutically acceptable salts, their enantiomers, the salts of their enantiomers, and mixtures thereof.
